(19) United States
(12) Patent Application Publication
(10) Pub. No.: US 2026/0284088 A1
(43) Pub. Date: Sep. 24, 2026

(54) COMPOSITION FOR IMPROVING METABOLIC FUNCTION

(71) Applicant: Spartacus Brands LLC, Boca Raton, FL (US)

(72) Inventors: Augustin Quancard, Boca Raton, FL (US); Gregory Papigny, Boca Raton, FL (US); Jeremy Eisenberg, Boca Raton, FL (US); Stephanie Eisenberg, Boca Raton, FL (US); Gary Bitton, Boca Raton, FL (US)

(21) Appl. No.: 19/086,726

(22) Filed: Mar. 21, 2025

(51) Int. Cl. A61K 31/7004; A23L 33/00; A61K 9/00

(52) U.S. Cl. CPC A61K 31/7004; A23L 33/40; A61K 9/0095

(57) ABSTRACT

The present invention is directed to composition for a nutritional supplement. The supplement comprises an allulose, and a nutrient mix comprising vitamins, minerals, and certain macronutrients. In some embodiments, the supplement further comprises a meal replacement mix. The allulose, nutrient mix, and meal replacement mix may be variously provided in effective amounts to improve metabolic function and control appetite in a human subject when ingested. In an embodiment, the essential nutrient mix comprises vitamin D3, magnesium, potassium, Omega-3 and vitamin K2. The meal replacement mix comprises protein, collagen, dietary fiber, and additional vitamins and minerals, while the vitamin

### GOVERNMENT CONTRACT

Not applicable.

### CROSS-REFERENCE TO RELATED APPLICATIONS

Not applicable.

### STATEMENT RE. FEDERALLY SPONSORED RESEARCH/DEVELOPMENT

Not applicable.

### COPYRIGHT & TRADEMARK NOTICES

A portion of the disclosure of this patent document may contain material which is subject to copyright protection. This patent document may show and/or describe matter which is or may become trade dress of the owner. The copyright and trade dress owner has no objection to the facsimile reproduction by any one of the patent document or the patent disclosure, as it appears in the Patent and Trademark Office patent files or records, but otherwise reserves all copyrights and trade dress rights whatsoever.

### TECHNICAL FIELD

The disclosed subject matter relates generally to a nutritional supplement to improve metabolic function and control appetite when ingested.

### BACKGROUND

Dietary supplements are intended to enhance the diet by supplying essential nutrients that may be lacking in an individual's daily consumption. These supplements can contain vitamins, minerals, amino acids, herbs, and other beneficial substances. With the increasing global emphasis on health and wellness, there is a growing demand for supplements that claim specific health benefits, including but not limited to weight management, metabolic health, and overall well-being enhancement.

Numerous food and beverage items contain nutritive sweeteners such as sucrose (commonly known as ‘sugar’ or ‘table sugar’), glucose, fructose, corn syrup, high fructose corn syrup, and similar ingredients. Despite their favorable taste and functional attributes in cooking, excessive consumption of these sweeteners, particularly sucrose, has long been linked to a rise in diet-related health issues, including obesity, cardiovascular disease, metabolic disorders, and dental issues. This troubling trend has led consumers to increasingly acknowledge the importance of adopting a healthier lifestyle and reducing their consumption of sweeteners with nutritional value.

In recent years, there has been a movement towards developing replacements for traditional sweeteners. One such sweetener is allulose, chemically known as D-psicose. Allulose is a rare sugar found naturally in small quantities in certain fruits like figs, raisins, and jackfruit. It is categorized as a “low-calorie sugar” or “rare sugar” due to its unique properties. Allulose has gained popularity as a sugar substitute because it has a taste similar to table sugar (sucrose) but with significantly fewer calories. Specifically, allulose provides approximately 0.2 calories per gram compared to 4 calories per gram for sucrose, making it effectively a “zero-calorie” sweetener. These properties make allulose an attractive option for inclusion in dietary supplements designed to support metabolic health, control cravings, manage weight and diabetes, decrease sugar consumption, act as a Glucagon-like peptide 1 (GLP-1) booster, and regulate blood sugar levels.

The use of allulose to promote health is discussed in PCT Pub. No. WO2017/029245 to Koch. The publication discloses the use of allulose as a prebiotic, showing how it can suppress the growth or activity of gut bacteria and improve intestinal flora balance. U.S. Pub. No. 2020/0170269 to Lee, discloses an allulose-containing composition for promoting lipid excretion. However, the role of allulose in managing cravings, controlling weight, or serving as a meal replacement—whether through daily consumption or as part of a food or dietary supplement—is not addressed.

Other products containing levels of allulose have been made in an attempt to provide food and beverage products with the desired bulking, sweetening, and functional properties traditionally provided by nutritive sweeteners, U.S. Pub, No. 2023/0248038 and U.S. Pub. No. 2016/0302463 to Woodyer, disclose the use of allulose syrups, their use in manufacturing food or beverage products, and their use as a sugar substitute in baking. U.S. Pub. No. 2018/0368457 to Pandey discloses a low calorie, low glycemic index (GI) sugar composition for use in food and beverage, U.S. Pat. No. 11,812,769 to Shim and U.S. Pub. No. 2021/0298341 to Han, disclose the use of allulose in beverages and nutritional drinks, respectively. U.S. Pub. No. 2022/0408778 to Bahar, discussed allulose combined with soluble high-fiber and low-sugar dietary fibers for various foods and beverage applications. Yet, the use of allulose as a dietary, nutritional, or daily supplement is not discussed.

Furthermore, U.S. Publication No. 2016/0331014 to Perera, discusses a solid food product with D-allulose syrup and native protein, aimed for the use of the solid food product as a protein supplement for dietitians, athletes, and bodybuilders. Additionally, it discusses D-allulose's role in preventing protein hardening, aiding hydration, and improving dough cohesiveness. However, this solid food product containing allulose syrup is not designed for the general population.

In an attempt to develop a replacement for a nutritive sweetener, U.S. Pat. No. 11,766,062 to Boit, discloses attributes of a D-allulose syrup and its ability to crystallize more efficiently than previous alternatives and its use in food and pharmaceutical products. U.S. Pub. No. 2022/0211090 to Icoz, discloses binding syrup with allulose and low sugar compositions for formulating food products. U.S. Pub. No. 2022/0144878 to Koch discloses an aqueous allulose liquid composition for use in food and beverages. Still, the use of allulose and liquid composition containing allulose in dietary, nutritional, or daily supplements is not discussed.

The utilization of allulose for health applications approached from a medical standpoint is discussed in U.S. Pat. No. 11,318,152. This patent discloses the use of D-allulose in a pharmaceutical agent aimed at improving autism spectrum disorder, enhancing social and mental behaviors, and also providing anti-anxiety and anti-stress effects. Nonetheless, this pharmaceutical agent is not meant for widespread adoption and does not fall under the categories of dietary, nutritional, or daily supplements.

While allulose shows promise as a health-promoting ingredient, its use as a dietary supplement is still emerging. The market for dietary supplements is diverse, with many products incorporating various ingredients to target specific health outcomes. However, there remains a need for supplement formulations that harness the unique benefits of allulose, addressing gaps in current offerings and providing a new option for all consumers seeking to enhance their metabolic health and overall well-being.

The present invention seeks to address these needs by providing novel supplement formulations that utilize allulose as a key ingredient. By harnessing the metabolic benefits of allulose, the invention aims to provide consumers with a dietary supplement that promotes health and wellness in a safe, effective, and innovative way.

Thus, although various proposals have been made to solve the problem(s), none of those in existence combine the characteristics of the present invention. Therefore, there remains a need for a nutritional sup that effectively leverages the metabolic benefits of allulose to support weight management, regulate blood sugar levels, reduce cravings, and promote overall metabolic health and well-being.

### SUMMARY

The present disclosure is directed to nutritional supplements containing allulose and for improving metabolic health and function. Some embodiments of the nutritional supplements comprise combinations of supplemental, essential micro and macronutrients, such as certain vitamins and minerals and fatty acids, and protein with a craving-control component as allulose and/or fiber. The composition may be provided in effective amounts to activate glucagon-like-peptide-1 (“GLP-1”) hormone receptors and in turn promote metabolic health, enhance nutritional balance, and support appetite suppression.

For purposes of summarizing, certain aspects, advantages, and novel features have been described. It is to be understood that not all such advantages may be achieved in accordance with any one particular embodiment. Thus, the disclosed subject matter may be embodied or carried out in a manner that achieves or optimizes one advantage or group of advantages without achieving all advantages as may be taught or suggested.

In some embodiments, the composition comprises effective amounts of supplemental allulose to activate GLP-1 receptors in combination with a plurality of additional essential nutrients known to those of ordinary skill in the art as being required for the body to function properly yet not sufficiently produced by the body on its own. These can include certain micro and micronutrients such as vitamins, minerals, fats, proteins, and other essential nutrients which may be lacking in a user's diet. Thus, in accordance with some embodiments, the composition is provided as allulose formulated together with the following exemplary essential nutrients: vitamin D, magnesium, potassium, fatty acids, and vitamin K2. In some embodiments, the nutrient mix may be vegan and comprise nutrients derived from non-animal sources so as to be suitable for ingestion by consumers adhering to plant based diets for any variety of reasons.

Embodiments of the composition may comprise a variety of essential nutrients, as in the following examples. Vitamin D may be provided as vitamin D2 or D3-ergocalciferol and cholecalciferol-respectively and derived from any appropriate animal or plant based source as needed or desired. In addition, embodiments of the composition may comprise macronutritional fatty acids, such as omega-3 and omega-6 fatty acids. These may be extracted from fatty fish like salmon, mackerel, sardines, anchovies, and tuna; krill; algae; and/or seeds such as flax seeds, chia seeds, hemp seeds, and walnuts. In some embodiments, the fatty acids are omega-3 fatty acids derived, for example only and not limitation, from wild-caught small fish. In some embodiments, the fatty acids may be omega-3 fatty acids provided in a 1:1 ratio of docosahexaenoic Acid (DHA) and eicosapentaenoic Acid (EPA). Additional vitamins, such as vitamin K as, for example only and not limitation, vitamin K2 (or MK-7) derived from animal products and/or fermentation, may also be included. Additional nutrients, such as potassium as potassium citrate, and any type of magnesium may be provided as well. In addition to providing fundamental micronutrients, the composition incorporates effective amounts of allulose to activate naturally occurring GLP-1 hormone receptors in an individual's body. Allulose, a non-nutritive sweetener with a sugar-like taste, helps manage appetite without adding caloric load, thereby aiding individuals in resisting highly palatable, nutrient-deficient foods prevalent in modern diets.

The allulose may be derived or sourced from plant matter by any acceptable manner known to those of ordinary skill in the art or later developed. For instance, the allulose may be produced through an enzymatic conversion process involving fructose-containing carbohydrates derived from, as non-limiting examples only and not limitation, corn or sugar beet, and using enzymes to convert the fructose into allulose by modifying its molecular structure.

In some embodiments, allulose and nutrient mix composition may be formulated for ingestion on a daily basis, up to once, twice, or even more times per day. It is contemplated that the dosage and number of times ingested by users per day, and indeed, the time of day taken and whether taken with additional food, supplements, or liquid, may depend on individual needs and preferences, determined by, for example only and not limitation, each individual's diet, weight, lifestyle, and existing supplementation practices and protocols. It is contemplated, however, that the composition may be provided as a dietary supplement ingested with one or more meals, and in particular, with an individual's first meal of the day, or breakfast.

Thus, in some embodiments, the composition may comprise supplemental allulose up to about 10 grams as well as a up to about 1 grams, up to about 2 grams or up to about 3 grams nutrient mix. As one particular example, an embodiment of the composition may comprise about 85 to about 95% by weight allulose and about 5 to about 15% by weight nutrient mix. In some embodiments, the nutrient mix alone comprises up to about 0.001% by weight vitamin D, up to about 4% by weight magnesium; up to about 2% by weight potassium citrate; up to about 5% by weight Omega-3; and up to about 0.01% by weight Vitamin K2.

In some embodiments, the nutritional supplement may comprise effective amounts of supplemental allulose to stimulate GLP-1 hormone receptors together with fiber, such as soluble fiber from, for example only and not limitation, psyllium husk. The nutritional supplement may be provided in amounts effective to stimulate GLP-1 hormone receptors, avoiding the insulin stimulation and blood sugar elevation commonly associated with nutritive sweeteners like high fructose corn syrup. The allulose may be combined with effective amounts of fiber to reduce food cravings, regulate appetite, and enhance digestion. Administering an effective dose of the fiber alongside a meal or as part of a sweetener supplement may effectively promote satiety, mitigate post-meal blood sugar spikes-particularly from carbohydrate-rich foods such as bread, crackers, pasta, and rice- and minimize excessive insulin production. In some embodiments, the allulose and fiber may be provided with or added to meals and/or drinks as a supplemental or replacement sweetener. Such composition may comprise as little as 1 gram, 2 grams, 3 grams, 4 grams, or even 5 grams of each of allulose and soluble fiber and up to about 10 grams of each of allulose and fiber per serving in powder, liquid, or capsule form. More particularly, some embodiments of the nutritional supplement may be 50 to about 55% by weight allulose; and 50 to about 55% by weight soluble fiber.

Some embodiments of the nutritional supplement may be further configured as a meal replacement powder or liquid taken as needed or desired. In such embodiments, the composition may comprise allulose, fiber, and the nutrient mix as described above, as well as a meal replacement mix as a combination of protein and collagen in effective amounts to improve satiety, reduce and even prevent cravings, and activate GLP-1 hormone receptors. In some embodiment, each serving of the exemplary composition may contain about 20 to about 30 grams of protein. Thus, in some embodiments, the nutritional supplement may comprise about 25 to about 35% by weight protein, 10 to about 20% by weight collagen, 10 to about 20% by weight allulose, 20 to about 25% by weight dietary fiber, and about 10 to about 20% by weight nutrient mix. The protein and collagen may be derived by any process and from any source suitable for supplementation without departing from the invention. As such, the protein may be any of whey, casein, egg or other animal-based proteins as well as soy, pea, rice, hemp or any other plant proteins as needed or desired based on dietary preferences and needs, performance in the composition, and even flavor preferences.

In plant based embodiments of the compositions herein described, animal based collagen may be eliminated without departing from the invention.

It is contemplated that providing a nutritional supplement according to the disclosure and claims provided below may effectively address modern nutritional challenges by combining effective, supplemental amounts of nutrients and fiber with allulose for controlling cravings.

Thus, it is an object of the invention to effectively address critical nutritional gaps caused by modern agricultural practices, soil depletion, and the prevalence of processed food.

It is another object of the invention to support users' efforts to maintain a balanced diet, by providing effective quantities of essential nutrients as a dietary supplement.

It is yet another object of the invention to simplify nutritional supplementation by delivering key nutrients in bioavailable forms in effective quantities.

It is still another object of the invention to combine essential nutrient supplementation with appetite suppression in a single ingestible product.

It is yet still another object of the invention to support metabolic health holistically by effectively supplement nutritional deficiencies and improve behavioral aspects of food consumption,

It is another object of the invention to deliver foundational nutritional support while moderating user cravings, increasing user satiety, and promoting overall user well-being in a user-friendly format.

One or more of the above-disclosed embodiments, in addition to certain alternatives, are provided in further detail below with reference to the attached figures. The disclosed subject matter is not, however, limited to any particular embodiment disclosed.

### BRIEF DESCRIPTION

The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Furthermore, the terms “include,” and “have,” and any variations thereof, are intended to cover a non-exclusive inclusion, such that a process, method, system, article, device, or apparatus that comprises a list of elements is not necessarily limited to those elements, but may include other elements not expressly listed or inherent to such process, method, system, article, device, or apparatus

The terms “couple,” “coupled,” “couples,” “coupling,” and the like should be broadly understood and refer to connecting two or more elements or signals, electrically, mechanically or otherwise. Two or more electrical elements may be electrically coupled, but not mechanically or otherwise coupled; two or more mechanical elements may be mechanically coupled, but not electrically or otherwise coupled; two or more electrical elements may be mechanically coupled, but not electrically or otherwise coupled. Coupling (whether mechanical, electrical, or otherwise) may be for any length of time, e.g., permanent or semi-permanent or only for an instant.

### DETAILED DESCRIPTION

Having summarized various aspects of the present disclosure, reference will now be made in detail to various embodiments of the composition. While the disclosure will be described in connection with these embodiments, there is no intent to limit it to the embodiment or embodiments disclosed herein. Rather, the intent is to cover all alternatives, modifications and equivalents included within the spirit and scope of the disclosure as defined by the appended claims.

With reference to It should be emphasized that the above-described embodiments are merely examples of possible implementations. Many variations and modifications may be made to the above-described embodiments without departing from the principles of the present disclosure. All such modifications and variations are intended to be included herein within the scope of this disclosure and protected by the following claims.

Moreover, embodiments and limitations disclosed herein are not dedicated to the public under the doctrine of dedication if the embodiments and/or limitations: (1) are not expressly claimed in the claims; and (2) are or are potentially equivalents of express elements and/or limitations in the claims under the doctrine of equivalents.

### EXAMPLES OF THE PREFERRED EMBODIMENT OF THE COMPOSITION

In order to more fully teach what the Applicant regards as their invention, the following example is given. It should be understood that the formulation set forth in the Examples are not to be construed as limiting the scope of the invention, except so far as they yield a composition having the desired properties and characteristics. More particularly, and by way of example, the following charts illustrate formulations of the same with percentages given by weight of the composition.

EXAMPLE 1 (Nutrient Mix and Allulose)

| Ingredient | Percentage |
|---|---|
| Allulose | 90.49822 |
| Cholecalciferol | 0.00068 |
| Magnesium | 3.17000 |
| Potassium citrate | 1.81000 |
| Omega-3 | 4.52000 |
| Vitamin K2 (MK-7) | 0.00110 |
| TOTAL: | 100.00000 |

EXAMPLE 1A (Nutrient Mix alone)

| Ingredient | Percentage |
|---|---|
| Cholecalciferol | 0.0071 |
| Magnesium | 33.3300 |
| Potassium citrate | 19.0400 |
| Omega-3 | 47.6115 |
| Vitamin K2 (MK-7) | 0.0114 |
| TOTAL: | 100.0000 |

EXAMPLE 2

| Ingredient | Percentage |
|---|---|
| Allulose | 15.14 |
| Whey | 30.28 |
| Collagen | 15.14 |
| Psyllium Husk | 22.71 |
| Essential Nutrient Mix | 16.73 |
| TOTAL: | 100.00 |

EXAMPLE 3

| Ingredient | Percentage |
|---|---|
| Allulose | 50.0 |
| Psyllium Husk | 50.0 |
| TOTAL: | 100.00 |

### CONCLUSIONS, RAMIFICATIONS, AND SCOPE

While certain embodiments of the invention have been illustrated and described, various modifications are contemplated and can be made without departing from the spirit and scope of the invention. For example, flavorings may be added as desired or needed to make the compositions more palatable. Accordingly, it is intended that the invention not be limited, except as by the appended claim(s).

The teachings disclosed herein may be applied to other systems, and may not necessarily be limited to any described herein. The elements and acts of the various embodiments described above can be combined to provide further embodiments. All of the above patents and applications and other references, including any that may be listed in accompanying filing papers, are incorporated herein by reference. Aspects of the invention can be modified, if necessary, to employ the systems, functions and concepts of the various references described above to provide yet further embodiments of the invention.

Particular terminology used when describing certain features or aspects of the invention should not be taken to imply that the terminology is being refined herein to be restricted to any specific characteristics, features, or aspects of the composition for improving metabolic function with which that terminology is associated. In general, the terms used in the following claims should not be constructed to limit the composition for improving metabolic function to the specific embodiments disclosed in the specification unless the above description section explicitly define such terms. Accordingly, the actual scope encompasses not only the disclosed embodiments, but also all equivalent ways of practicing or implementing the disclosed system, method and apparatus. The above description of embodiments of the composition for improving metabolic function is not intended to be exhaustive or limited to the precise form disclosed above or to a particular field of usage.

While specific embodiments of, and examples for, the method, system, and apparatus are described above for illustrative purposes, various equivalent modifications are possible for which those skilled in the relevant art will recognize.

While certain aspects of the method and system disclosed are presented below in particular claim forms, various aspects of the method, system, and apparatus are contemplated in any number of claim forms. Thus, the inventor reserves the right to add additional claims after filing the application to pursue such additional claim forms for other aspects of the composition for improving metabolic function.

What is claimed is:

1. A nutritional supplement, comprising:
allulose and a nutrient mix,
wherein the nutritional supplement is operative improve metabolic function and control appetite in a human subject when ingested by such subject.

2. The nutritional supplement of claim 1, comprising:
85% by weight to about 95% by weight allulose; and
5% by weight to about 15% by weight nutrient mix;
wherein the nutritional supplement is operative to improve metabolic function and control appetite in a human subject when ingested by such subject.

3. The nutritional supplement of claim 2, wherein the nutrient mix comprises a combination of vitamin D3, magnesium, potassium, Omega-3, and Vitamin K2.

4. The nutritional supplement of claim 3, wherein the nutrient mix comprises,
up to about 0.001% by weight vitamin D3 as cholecalciferol;
up to about 4% by weight magnesium;
up to about 2% by weight potassium citrate;
up to about 5% by weight Omega-3; and
up to about 0.01% by weight Vitamin K2.

5. The nutritional supplement of claim 2, comprising the allulose and nutrient mix in a unit dosage effective to activate GLP-1 receptors.

6. The nutritional supplement of claim 2 comprising the allulose and nutrient mix in a unit dosage of between about 10 g to about 15 g.

7. The nutritional supplement of claim 1, further comprising a meal replacement mix, the meal replacement mix comprising protein, collagen, and dietary fiber.

8. The nutritional supplement of claim 6, further comprising a meal replacement mix,
25 to about 35% by weight protein;
10 to about 20% by weight collagen;
10 to about 20% by weight allulose;
20 to about 25% by weight dietary fiber; and
10 to about 20% by weight additional vitamins and minerals;
wherein the nutritional supplement comprising all of the allulose, the nutrient mix, and meal replacement mix is composed as an effective, nutritive meal replacement.

9. The nutritional supplement of claim 6, wherein the protein is a complete protein or combination of protein sources defining a complete protein.

10. The nutritional supplement of claim 8, wherein the complete protein is whey.

11. The nutritional supplement of claim 7, comprising the protein, collagen, allulose, dietary fiber, and additional vitamins and minerals in a dosage unit effective to activate GLP-1 receptors.

12. The nutritional supplement of claim 7, comprising the protein, collagen, allulose, dietary fiber, and additional vitamins and minerals in a unit dosage of between about 40 g to about 60 g.

13. A human food supplement, comprising:
50 to 55% by weight allulose; and
50 to 55% by weight soluble fiber;
wherein the food supplement is non-nutritive and operative to stimulate the release of GLP-1, improve metabolic function, and control appetite in a human subject when ingested by such subject.

14. The human food supplement of claim 13, comprising the allulose and soluble fiber in a unit dosage of between about 5 g to about 20 g.

15. A method of improving metabolic function and controlling appetite in a human subject, comprising:
providing to a human subject an effective amount of allulose to activate GLP-1 hormone receptors, wherein the allulose is provided as a dietary supplement.

16. The method of claim 15, further comprising providing the effective amount of allulose together with fiber.

17. The method of claim 15, further comprising providing the effective amount of allulose together with a nutrient mix comprising vitamin D3 as cholcalciferol, magnesium, potassium citrate, Omega-3, Vitamin K2.

18. The method of claim 16, further comprising providing the effective amount of allulose together with the nutrient mix as well as a meal replacement mix comprising protein, fiber, and collagen.

19. The method of claim 17, wherein the protein is a complete protein.

20. The method of claim 14, wherein the providing step is by ingestion.
